(19) United States
(12) Patent Application Publication
(10) Pub. No.: US 2022/0397575 A1
(43) Pub. Date: Dec. 15, 2022

(54) METHODS FOR DETERMINING THE LIKELIHOOD OF LUNG CANCER

(71) Applicant: National University of Singapore, Singapore (SG)

(72) Inventors: Boon Cher GOH, Singapore (SG); Lingzhi WANG, Singapore (SG); Sok Hwee Esther CHEOW, Singapore (SG); Henry YANG, Singapore (SG)

(21) Appl. No.: 17/621,407

(22) PCT Filed: Jun. 19, 2020
(86) PCT No.: PCT/SG2020/050343

§ 371 Date: Dec. 21, 2021

(30) Foreign Application Priority Data
Jun. 21, 2019 (SG) 10201905757Q

(51) Int. Cl. G01N 33/574

(52) U.S. Cl. CPC G01N 33/57423; G01N 33/57484

(57) ABSTRACT

The present invention is directed to a method of determining the likelihood of the presence of lung cancer in a subject. The method comprising determining the level of a biomarker selected from the group consisting of catalase (CAT), C-X-C motif chemokine receptor 4 (CXCR4), superoxide dismutase 3 (SOD3) and surfactant protein B (SFTPB) from a vesicle population isolated from a biological sample from said subject, wherein a change in the level of biomarker as compared to a reference indicates the likelihood of the presence of lung cancer in the subject.

### FIELD OF INVENTION

The invention relates generally to the field of biotechnology. In particular, the disclosure relates to a method of determining the likelihood of the presence of lung cancer in a subject and methods of treating lung cancer in a subject.

### BACKGROUND

Lung cancer is the second most common type of cancer diagnosed in the United States, with about 234,030 new cases in United States in 2018. It is the leading cause of cancer death among both men and women. Lung cancer is mainly diagnosed in older people, with an average age of about 70.

Current lung screening options includes chest x-ray, sputum cytology and chest computed tomography (CT), each with their respective weighted merits and limitation. Among these approaches, sputum cytology is a non-invasive method but has very poor detection rate. For chest x-ray, it also shows a low sensitivity and specificity for early detection of lung cancer. Even though low-dose CT has a high sensitivity but it has been demonstrated to have an extremely poor specificity, resulting in about 96% false positive rate. Majority of patients detected by low-dose CT have been demonstrated as false positive lung cancer through invasive biopsies. Therefore, the clinical usefulness of these costly invasive tools remained controversial and unsatisfactory in the facilitation of early lung cancer detection and intervention to improve mortality, in-part due to the high incidence benign nodules, making interpretation extremely challenging.

Accordingly, it is generally desirable to overcome or ameliorate one or more of the above mentioned difficulties.

### SUMMARY

Disclosed herein is a method of determining the likelihood of the presence of lung cancer in a subject, the method comprising determining the level of a biomarker selected from the group consisting of catalase (CAT), C-X-C motif chemokine receptor 4 (CXCR4), superoxide dismutase 3 (SOD3) and surfactant protein B (SFTPB) from a vesicle population isolated from a biological sample from said subject, wherein a change in the level of biomarker as compared to a reference indicates the likelihood of the presence of lung cancer in the subject.

Also disclosed herein is a method of determining the progression of lung cancer in a subject, the method comprising determining the level of a biomarker selected from the group consisting of CAT, CXCR4, SOD3 and SFTPB from a vesicle population isolated from a biological sample from said subject, wherein a change in the level of biomarker as compared to a reference distinguishes between the presence of early stage and late stage lung cancer in a subject.

Also disclosed herein is a method of detecting and treating lung cancer in a subject, the method comprising: (a) determining the level of a biomarker selected from the group consisting of CAT, CXCR4, SOD3 and SFTPB in a vesicle population isolated from a biological sample from said subject, wherein a change in the level of biomarker as compared to a reference indicates the presence of lung cancer in the subject, and (b) administering an anti-cancer therapy to the subject.

Also disclosed herein is a method of monitoring the responsiveness of a subject suffering from lung cancer to an anti-cancer therapy, the method comprising determining the level of a biomarker selected from the group consisting of CAT, CXCR4, SOD3 and SFTPB in a vesicle population isolated from a biological sample from said subject, wherein (a) an increase or no change in the level of biomarker as compared to a reference indicates that the subject is non-responsive to the anti-cancer therapy, and wherein (b) a decrease in the level of biomarker as compared to a reference indicates that the subject is responsive to the anti-cancer therapy.

### BRIEF DESCRIPTION OF DRAWINGS

Embodiments of the present invention are hereafter described, by way of non-limiting example only, with reference to the accompanying drawings in which:

FIG. 1: Exosome biomarker discovery workflow

FIG. 2: Characterisation of exosomes from human plasma (200 μl) isolated from ultracentrifugation (UC) and total exosome isolation kit (Invitrogen). (A) Respective electron micrographs of plasma exosomes enriched from pooled healthy donors (n=33), low magnification (80,000×, scale bar=500 nm). Insert shows a magnified portion of micrograph (200,000×, scale bar=200 nm). (B) Concentration (particles/ml) and size distribution (nm) profile of respective exosomes-enriched preparations from pooled healthy donors by NTA. (C) Respective immunoblot analyses of pooled plasma exosomes and pooled exosome-depleted plasma against four positive (+) exosome markers, and two negative (−) exosome markers. Transferrin (TF) served as loading control. Healthy, n=33; NSCLC Early, n=13; NSCLC Late, n=60.

FIG. 3. Assessment of quantitative MS data quality. (A) Venn diagram of overlapping proteins quantified in three TMT technical replicates (B) Correlation assessment between quantitative dataset. Scatter log 2 plots of measured ratios for each protein between triplicates.

FIG. 4. Immunoblot verification of plasma exosomes. Normalized mean densitometry values (log 2) of six candidate exosome biomarkers in early-stage NSCLC (n=14); late-stage NSCLC (n=14) and healthy individuals (n=14) as shown in box-and-whiskers plots. Line represents median, box represents 25th to 75th percentiles and whisker represent maximum and minimum ranges and dot represent individual values. One-way ANOVA comparison between control and NSCLC phenotypes. Densitometry values are normalized against transferrin (Loading control). Abbreviation(s): **, p<0.01; ***, p<0.001; ****, p<0.0001.

FIG. 5. Receiver operating characteristic curve (ROC). (A) ROC analyses of exosome protein candidates and clinically in-used markers to differentiate NSCLC phenotypes from healthy subjects. (Healthy, n=167; Early NSCLC, n=64; All NSCLC, n=357). (B) ROC analyses of exosome protein candidates and clinically in-used markers to differentiate cancer phenotypes from healthy subjects. (Healthy, n=167; Breast, n=113; Colorectal, n=144; Nasopharyngeal, n=101).

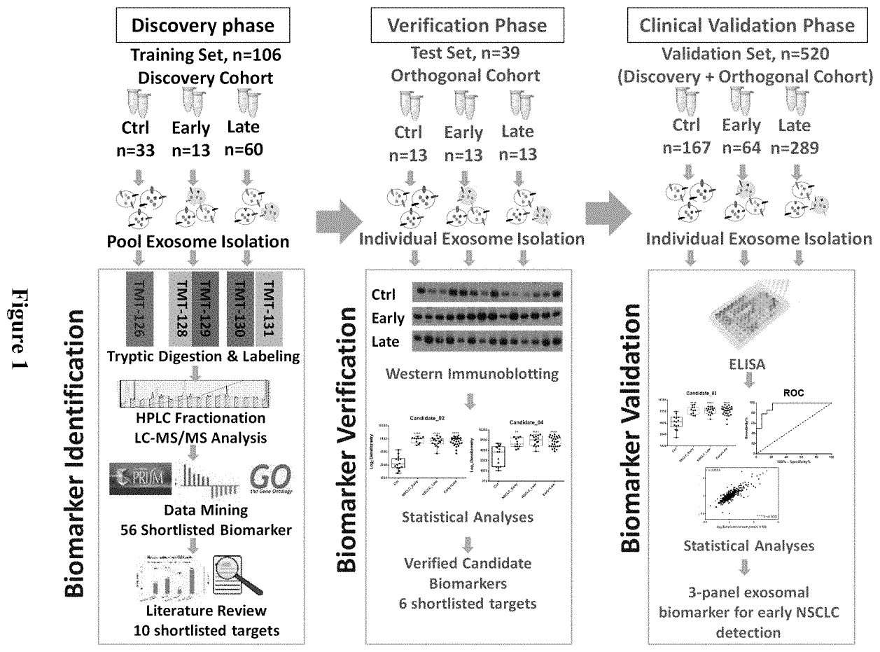

### DETAILED DESCRIPTION

Disclosed herein is a method of determining the likelihood of the presence of lung cancer in a subject, the method comprising determining the level of a biomarker selected from the group consisting of CAT, CXCR4, SOD3 and SFTPB from a vesicle population isolated from a biological sample from said subject, wherein a change in the level of biomarker as compared to a reference indicates the likelihood of the presence of lung cancer in the subject. In one embodiment, the method determines the likelihood of the presence of both early stage and/or late stage lung cancer in the subject.

Disclosed herein is a method of determining the likelihood of the presence of lung cancer in a subject, the method comprising determining the level of a biomarker selected from the group consisting of CAT, CXCR4, SOD3 and SFTPB from a vesicle population isolated from a biological sample from said subject, wherein an increase in the level of biomarker as compared to a reference indicates the likelihood of the presence of lung cancer in the subject.

Disclosed herein is a method of detecting the presence of lung cancer in a subject, the method comprising determining the level of a biomarker selected from the group consisting of CAT, CXCR4, SOD3 and SFTPB from a vesicle population isolated from a biological sample from said subject, wherein a change in the level of biomarker as compared to a reference indicates the presence of lung cancer in the subject. In one embodiment, the method detects the presence of early and/or late stage lung cancer in the subject.

Disclosed herein is a method of detecting the presence of lung cancer in a subject, the method comprising determining the level of a biomarker selected from the group consisting of CAT, CXCR4, SOD3 and SFTPB from a vesicle population isolated from a biological sample from said subject, wherein an increase in the level of biomarker as compared to a reference indicates the presence of lung cancer in the subject.

Without being bound by theory, the inventors have found that with exosomes, one can look at a well-defined entity in the blood, having all the advantages of blood samples but without background (fluctuations of marker proteins due to other disease or injury) and interference from plasma proteins. Since tumor cells are known to release much more circulatory exosomes than normal proliferating cells, this approach optimally supports the direct interrogation of NSCLC tumour-derived exosomes in plasma.

The three or four-marker exosome panel may, for example, be used in annual health screening, as well as before imaging to define high-risk patients. Those patients with a high-risk clinical profile in combination with the panel can proceed on to a chest computed tomography (CT). Those whose test results suggest a low probability of cancer can be re-evaluated with the plasma markers during their routine follow-up. Without being bound by theory, the point of care diagnostic panel of the present invention can greatly reduce false positive cases (˜50%) associated with screening CT, that may result in unnecessary anxiety, biopsies and/or surgery; and early detection will allow for timely tailored therapeutic strategies in the management, and improvement of NSCLC prognosis.

In one embodiment, the method is an in vitro or ex vivo method.

The phrase “likelihood of the presence of lung cancer” refers to how likely it is for a lung cancer to be present in a subject. An increase in the level of one or more biomarkers as compared to a reference may indicate a likelihood (i.e. chance or risk) of the presence of lung cancer in the subject. This could be, for example, a more than 10%, 20%, 30%, 40%, 50%, 60%, 70% 80%, 90% or 99% likelihood of the presence of lung cancer in the subject.

In one embodiment, there is provided a method of determining the likelihood of the presence of lung cancer in a subject, the method comprising determining the level of a vesicle-associated (or bound) biomarker selected from the group consisting of CAT, CXCR4, SOD3 and SFTPB in a biological sample from said subject, wherein a change (or increase) in the level of biomarker as compared to a reference indicates the likelihood of the presence of lung cancer in the subject.

The biomarker may be a protein, peptide. The biomarker may be associated or bound to the surface of the vesicle. Alternatively, it may be contained within the vesicle. In an alternative embodiment, the biomarker is a nucleic acid.

In one embodiment, the level of the biomarker is determined using an antibody-based technique or a PCR-based technique. The biomarker may, for example, be detecting using antibody-based techniques such as enzyme-linked immunosorbent assay (ELISA), Luminex assay or Western Immunoblotting, to determine the amount of biomarker that is associated, bound or contained within vesicles. The antibody may be an antibody that is binds specifically to a biomarker selected from the group consisting of CAT, CXCR4, SOD3 and SFTPB. The antibody may be further conjugated to a detectable label (such as a fluorescent, luminescent or enzyme label) to allow detection. Alternatively, the antibody may be detected using a secondary antibody that is conjugated to a label (such as a fluorescent, luminescent or enzyme label).

In one embodiment, the level of the biomarker is determined using a PCR-based technique. Analysis of the vesicles can include RNA sequence analysis by methods known in the art. For example, vesicles can be lysed and the RNA retrieved for RT-PCR analysis. Methods to determine the mRNA level of a gene in a sample are well known in the art. For example, the mRNA level can be determined by PCR, qPCR, qRT-PCR, RNA-sequencing, microarray analysis, SAGE, MassARRAY technique, next-generation sequencing, or FISH. Alternatively, the captured vesicle, either on the capture surface, or released from the capture surface, can be analyzed using immunocytochemical and other fluorescent imaging techniques. Analysis of the vesicles can also include detecting the presence of DNA molecules using techniques known in the art, such as PCR analysis or genomic sequencing.

Other techniques such as flow cytometry may also be used. Alternatively, the biomarker may also be detected using mass-spectrometry. A nucleic acid biomarker (such as genomic DNA or mRNA) may also be detected using PCR-based techniques.

The terms “peptide”, “polypeptide” and “protein” are used interchangeably and include any polymer of amino acids (dipeptide or greater) linked through peptide bonds or modified peptide bonds, whether produced naturally or synthetically. The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

Nucleic acids of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

As used herein, the term “antibody” includes, but is not limited to, synthetic antibodies, monoclonal antibodies, recombinantly produced antibodies, multispecific antibodies (including bi-specific antibodies), human antibodies, humanized antibodies, chimeric antibodies, single-chain Fvs (scFv), Fab fragments, F(ab′) fragments, disulfide-linked Fvs (sdFv) (including bi-specific sdFvs), and anti-idiotypic (anti-Id) antibodies, and epitope-binding fragments of any of the above. The antibodies provided herein may be monospecific, bispecific, tri specific or of greater multi-specificity.

The term “Polymerase chain reaction” or “PCR” means a reaction for the in vitro amplification of specific nucleic acid sequences by the simultaneous primer extension of complementary strands of nucleic acid molecules. In other words, PCR is a reaction for making multiple copies or replicates of a target nucleic acid flanked by primer sites, such reaction comprising one or more repetitions of the following steps: (i) denaturing the target nucleic acid, (ii) annealing primers to the primer sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art. The term “PCR” encompasses derivative forms of the reaction, including but not limited to, Reverse transcription-PCR, real-time PCR, nested PCR, quantitative PCR, multiplexed PCR, and the like.

In one embodiment, the method comprises determining the level of a biomarker selected from the group consisting of CAT, CXCR4, SOD3 and SFTPB that is present in a vesicle population or is associated, bound or contained within vesicles. In one embodiment, the method comprises determining the level of CAT, CXCR4, SOD3 or SFTPB. The method may comprise determining the level of a panel of biomarkers (i.e. two or more biomarkers). In one embodiment, the method comprises determining the level of two biomarkers comprising i) CAT and CXCR4, ii) CAT and SOD3, iii) CAT and SFTPB, iv) CXCR4 and SOD3, v) CXCR4 and SFTPB, or vi) SOD3 and SFTPB. In one embodiment, the method comprises determining the level of three biomarkers comprising i) CAT, CXCR4 and SOD3, ii) CAT, CXCR4 and SFTPB, iii) CAT, SOD3 and SFTPB or iv) CXCR4, SOD3 and SFTPB. In one embodiment, the method comprises determining the level of three biomarkers comprising CAT, CXCR4 and SFTPB. In one embodiment, the method comprises determining the level of four biomarkers comprising CAT, CXCR4, SOD3 and SFTPB. In one embodiment, the method comprises determining the level of four biomarkers consisting of CAT, CXCR4, SOD3 and SFTPB.

In one embodiment, there is provided a method of determining the likelihood of the presence of lung cancer in a subject, the method comprising determining the level of biomarkers comprising CAT, CXCR4 and SFTPB from a vesicle population isolated from a biological sample from said subject, wherein a change (or increase) in the level of the biomarkers as compared to a reference indicates the likelihood of the presence of lung cancer in the subject. The method may further comprise determining the level of SOD3.

In one embodiment, there is provided a method of determining the likelihood of the presence of lung cancer in a subject, the method comprising determining the level of biomarkers comprising CAT, CXCR4, SOD3 and SFTPB from a vesicle population isolated from a biological sample from said subject, wherein a change (or increase) in the level of the biomarkers as compared to a reference indicates the likelihood of the presence of lung cancer in the subject.

The biomarkers as referred to herein may be used in combination with other biomarkers that are known in the art for determining the likelihood of the presence of lung cancer in a subject. These include CA125, CEA and/or Cyfra-21.

The method may comprise isolating the vesicle population from said biological sample. The vesicle population may be isolated using techniques that include ultracentrifugation, size exclusion chromatography, density gradient centrifugation, differential centrifugation, nanomembrane ultrafiltration, immunoabsorbent capture, affinity purification, affinity selection, microfluidic separation or a combination thereof.

In one embodiment, the vesicle population (or exosome population) may be isolated with differential centrifugation followed by ultracentrifugation (which is the gold standard method. Other methods of enrichment include density gradient centrifugation, size-exclusion chromatography, filtration techniques, polymer-based precipitation, immunological separation and isolation by sieving.

The method of the present invention may comprise isolating the vesicle population prior to measurement of the level of the biomarkers. The method may further comprise lysing the vesicle population prior to measurement of the level of the biomarkers. Methods of lysing vesicle populations are known in the art. For example, vesicle populations may be lysed using a lysis buffer such as radio-immunoprecipitation assay (RIPA) buffer.

In one embodiment, the method comprises detecting the vesicle population with an antibody. For example, this may comprise detecting a surface marker from an exosomal population. This will preferably allow for isolation, purification and/or enrichment of said exosomal population. For the purposes of the present invention, the term “isolation” and “isolating” in all their grammatical forms relate to the act of separating or recovering exosomes from their environment, e.g. a serum or plasma sample or a tissue biopsy. The terms “purifying” and “purification” in all their grammatical forms relate to the act of freeing the desired exosomes from (non-exosomal) contaminants. The terms “enriching” and “enrichment” in all their grammatical forms mean increasing the proportion of exosomes in their respective solvent(s). Proteins are particularly envisaged as exosomal surface markers, but other biomolecules such as lipids are also conceivable. The exosomal surface marker may be recognized by an antibody. In one embodiment, the antibody is selected from the group consisting of an anti-CD9 antibody, an anti-CD63 antibody and an anti-CD 81 antibody.

In one embodiment, the method comprises isolating the vesicle population with a bead-conjugated antibody (such as in an ELISA based assay). The bead-conjugated antibody allows any antibody-bound vesicle population to be separated from the biological sample using techniques such as centrifugation or magnetic separation (in cases where the bead is a magnetic bead) and optionally one or more washing steps. In one embodiment, the antibody is selected from the group consisting of an anti-CD9 antibody, an anti-CD63 antibody and an anti-CD81 antibody.

The biological sample obtained from the subject can be any bodily fluid. For example, the biological sample can be peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation which may be of fetal or maternal origin. The biological sample may also be a tissue sample or biopsy from which vesicles and other circulating biomarkers may be obtained.

For many diseases (such as many cancers), invasive tissue biopsies followed by histopathological or molecular analysis are considered as diagnostic gold standard. Regardless of whether such procedures are performed as highly invasive surgeries or as less invasive needle punctures, tissue biopsies carry the risk of infection and cannot be applied repeatedly. Moreover, core and needle biopsies often do not result in sufficient amounts of tissue for in depths diagnostic analyses and can even miss zonal pathophysiological tissue alterations. Because blood samples can be easily and repeatedly obtained, the concept of “liquid biopsies” has held promise as a less invasive complement to traditional tissue biopsies. Upon secretion into bodily fluids, vesicles can be isolated via ultracentrifugation from said liquid biopsies.

In one embodiment, the biological sample is a bodily fluid for liquid biopsy. In one embodiment, the biological sample is a blood, serum or plasma sample. In one embodiment, the biological sample comprises a cancer cell or a circulating tumor cell (CTC). In another embodiment, the biological sample comprises a vesicle from a cancer cell or a circulating tumor cell.

Methods of the invention can include assessing one or more vesicles, including assessing vesicle populations. As used herein, a “vesicle” may refer to a naturally occurring or synthetic vesicle that includes a cavity inside. The vesicle may comprise a lipid bilayer membrane enclosing contents of an internal cavity. A vesicle may include a liposome, an exosome, extracellular vesicle, microvesicle, apoptotic vesicles (or apoptotic body), a vacuole, a lysosome, a transport vesicle, a secretory vesicle, a gas vesicle, a matrix vesicle, or a multivesicular body. A vesicle may have a dimension of about 1000 nm or less, about 900 nm or less, about 800 nm or less, about 700 nm or less, about 600 nm or less, about 500 nm or less, about 450 nm or less, about 400 nm or less, about 350 nm or less about 300 nm or less, about 250 nm or less, about 240 nm or less, about 230 nm or less, about 220 nm or less, about 210 nm or less, about 200 nm or less, about 190 nm or less, about 180 nm or less, about 170 nm or less, about 160 nm or less, about 150 nm or less, about 140 nm or less, about 130 nm or less, about 120 nm or less, about 1 10 nm or less, about 100 nm or less, about 90 nm or less, about 80 nm or less, about 70 nm or less, about 60 nm or less, about 50 nm or less, about 40 nm or less, about 30 nm or less, about 20 nm or less, or about 10 nm or less.

Exosomes are a type of vesicle, also referred to in the art as extracellular vesicles, microvesicles or microparticles. These vesicles are shed by eukaryotic cells, or budded off of the plasma membrane, to the exterior of the cell. These membrane vesicles are heterogeneous in size with diameters ranging from about 10 nm to about 5000 nm. The small vesicles (approximately 10 to 1000 nm, preferably 30 to 100 nm in diameter) that are released by exocytosis of intracellular multivesicular bodies are referred to in the art as “exosomes”. The methods and compositions described herein are equally applicable for other vesicles of all sizes.

Structurally, exosomes can be described as spherical bilayered proteolipids carrying a cargo of various biomolecules, including genetic material such as mRNA, microRNA (miRNA), and other non-coding RNAs or even small amounts of DNA, lipids and proteins including transcription factors, cytokines, growth factors and others.

In one embodiment, the vesicle is an exosome. In another embodiment, the vesicle is a circulatory exosome.

As described above, vesicles can be either isolated from the sample (e.g. by utilizing surface markers which are bound by a suitable antibody) prior to further analysis or be analyzed directly from the sample (e.g. by detecting the level of one or more biomarkers as described herein). “Analysis” may, in general, include quantification of the amount vesicles in a sample and/or assessing the level of the one or more biomarkers indicative for lung cancer.

The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a sub-stage of a Stage II cancer. One skilled in the art will appreciate that the classification of a Stage II cancer as either an early stage cancer or a late stage cancer depends on the particular type of cancer. In one embodiment, the cancer is lung cancer. In one embodiment, the cancer is non-small cell lung cancer (NSCLC) or small cell lung cancer (SCLC).

In one embodiment, the method comprises treating the subject found to have lung cancer.

The method as defined herein may comprise the step of determining the level of a biomarker selected from the group consisting of CAT, CXCR4, SOD3 and SFTPB from a vesicle population isolated from a biological sample from said subject, wherein a change (increase) in the level of biomarker as compared to a reference indicates the presence of lung cancer in the subject.

The “reference” as referred to herein may be one or more non-cancerous samples taken from the same subject or one or more non-cancerous samples taken from another subject (e.g. a healthy subject who does not suffer from cancer). The reference may also be a pre-determined value or an average value. In one embodiment, the method as defined herein comprises the step of comparing the level of one or more biomarkers to a reference.

As used herein, the term “increase” or “increased” with reference to a biomarker refers to a statistically significant and measurable increase in the biomarker as compared to a reference. The increase may be an increase of at least about 10%, or an increase of at least about 20%, or an increase of at least about 30%, or an increase of at least about 40%, or an increase of at least about 50%.

As used herein, the term “decrease” or “decreased” with reference to a biomarker refers to a statistically significant and measurable decrease in the biomarker as compared to a reference. The decrease may be a decrease of at least about 10%, or a decrease of at least about 20%, or a decrease of at least about 30%, or a decrease of at least about 40%, or a decrease of at least about 50%.

In one embodiment, an increase in the level of a biomarker as compared to a reference may be an increase of 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 31 fold, 32 fold, 33 fold, 34 fold, 35 fold, 36 fold, 37 fold, 38 fold, 39 fold, 40 fold, 41 fold, 42 fold, 43 fold, 44 fold, 45 fold, 46 fold, 47 fold, 48 fold, 49 fold, 50 fold, 51 fold, 52 fold, 53 fold, 54 fold, 55 fold, 56 fold, 57 fold, 58 fold, 59 fold, 60 fold, 61 fold, 62 fold, 63 fold, 64 fold, 65 fold, 66 fold, 67 fold, 68 fold, 69 fold, 70 fold, 71 fold, 72 fold, 73 fold, 74 fold, 75 fold, 76 fold, 77 fold, 78 fold, 79 fold, 80 fold, 81 fold, 82 fold, 83 fold, 84 fold, 85 fold, 86 fold, 87 fold, 88 fold, 89 fold, 90 fold, 91 fold, 92 fold, 93 fold, 94 fold, 95 fold, 96 fold, 97 fold, 98 fold, 99 fold or 100 fold increase, or anywhere in between.

In one embodiment, an increase in one or more, two or more, three or more, or all four biomarkers as compared to a reference indicates the presence of lung cancer in the subject.

In one embodiment, a decrease in the level of a biomarker may refer to a biomarker having 0.9 times or less, 0.85 times or less, 0.8 times or less, 0.75 times or less, 0.7 times or less, 0.6 times or less, 0.55 times or less, 0.5 times or less, 0.45 times or less, 0.4 times or less, 0.35 times or less, 0.3 times or less, 0.25 times or less, 0.2 times or less, 0.15 times or less, 0.1 times or less or anywhere in between as compared to the level of a reference.

The invention is directed to a method of determining the progression of lung cancer in a subject. Provided herein is a method of determining the progression of lung cancer in a subject, the method comprising determining the level of a biomarker selected from the group consisting of CAT, CXCR4, SOD3 and SFTPB from a vesicle population isolated from a biological sample from said subject.

Also provided herein is a method of determining the progression of lung cancer in a subject, the method comprising determining the level of a biomarker selected from the group consisting of CAT, CXCR4, SOD3 and SFTPB from a vesicle population isolated from a biological sample from said subject, wherein a change in the level of biomarker as compared to a reference distinguishes between the presence of early stage and late stage lung cancer in a subject. The method may provide an indication of whether the lung cancer is a stage 0, I, II, III and/or IV cancer. In one embodiment, the method comprises determining the level of SOD3 from a vesicle population isolated from a biological sample from said subject.

Provided herein is a method of determining the progression of lung cancer in a subject, the method comprising determining the level of a biomarker selected from the group consisting of CAT, CXCR4, SOD3 and SFTPB from a vesicle population isolated from a biological sample from said subject, wherein an increase in the level of biomarker as compared to a reference distinguishes between the presence of early stage and late stage lung cancer in a subject.

The term “determining the progression of lung cancer” as used herein may refer to determining whether a lung cancer is an early stage or late stage cancer. It may also refer to whether the lung cancer is a stage 0, I, II, II and/or IV cancer.

Also provided herein is a method of determining the prognosis of lung cancer in a subject following an anti-cancer therapy, the method comprising determining the level of a biomarker selected from the group consisting of CAT, CXCR4, SOD3 and SFTPB from a vesicle population isolated from a biological sample from said subject.

Also disclosed herein is a method of detecting and treating lung cancer in a subject, the method comprising: (a) determining the level of a biomarker selected from the group consisting of CAT, CXCR4, SOD3 and SFTPB in a vesicle population isolated from a biological sample from said subject, wherein a change in the level of biomarker as compared to a reference indicates the presence of lung cancer in the subject, and (b) administering an anti-cancer therapy to the subject.

In one embodiment, there is provided a method of detecting and treating lung cancer in a subject, the method comprising: (a) determining the level of a biomarker selected from the group consisting of CAT, CXCR4, SOD3 and SFTPB in a vesicle population isolated from a biological sample from said subject, wherein an increase in the level of biomarker as compared to a reference indicates the presence of lung cancer in the subject, and (b) administering an anti-cancer therapy to the subject.

Also disclosed herein is a method of treating lung cancer in a subject. The method may be based on a test result that has been conducted by determining the level of a biomarker selected from the group consisting of CAT, CXCR4, SOD3 and SFTPB in a vesicle population isolated from a biological sample from said subject, wherein a change (or increase) in the level of biomarker as compared to a reference indicates the presence of lung cancer in the subject. The method of treating lung cancer may comprise administering an anti-cancer therapy to the subject.

Also disclosed herein is an anti-cancer therapy for use in the treatment of lung cancer in a subject, wherein the level of a biomarker selected from the group consisting of CAT, CXCR4, SOD3 and SFTPB in a vesicle population isolated from a biological sample from said subject has been determined, and wherein a change (or increase) in the level of biomarker as compared to a reference indicates the presence of lung cancer in the subject.

Also disclosed herein is a use of an anti-cancer therapy in the manufacture of a medicament for the treatment of lung cancer in a subject, wherein the level of a biomarker selected from the group consisting of CAT, CXCR4, SOD3 and SFTPB in a vesicle population isolated from a biological sample from said subject has been determined, and wherein a change (or increase) in the level of biomarker as compared to a reference indicates the presence of lung cancer in the subject.

The term “treating” or “treatment” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The term “administering” refers to contacting, applying or providing an anti-cancer therapy to a subject.

The term “subject” as used throughout the specification is to be understood to mean a human or may be a domestic or companion animal. While it is particularly contemplated that the methods of the invention are for treatment of humans, they are also applicable to veterinary treatments, including treatment of companion animals such as dogs and cats, and domestic animals such as horses, cattle and sheep, or zoo animals such as primates, felids, canids, bovids, and ungulates. The “subject” may include a person, a patient or individual, and may be of any age or gender.

In one embodiment, the method further comprises administering an anti-cancer therapy to the subject found to have lung cancer. The anti-cancer therapy may include chemotherapy, radiation therapy, a targeted therapy, immunotherapy, or a combination thereof. The chemotherapy may, for example, be cisplatin, carboplatin, paclitaxel (Taxol), albumin-bound paclitaxel (nab-paclitaxel, Abraxane), docetaxel (Taxotere), gemcitabine (Gemzar), vinorelbine (Navelbine), irinotecan (Camptosar), etoposide (VP-16), vinblastine or pemetrexed (Alimta). The method may also comprise treating the subject by surgery.

Also disclosed herein is a method of monitoring the responsiveness of a subject suffering from lung cancer to an anti-cancer therapy, the method comprising determining the level of a biomarker selected from the group consisting of CAT, CXCR4, SOD3 and SFTPB in a vesicle population isolated from a biological sample from said subject. A change in the level of biomarker (such as an increase or decrease) as compared to a reference may indicate that the subject is responsive to the anti-cancer therapy. In one embodiment, a decrease in the level of biomarker as compared to a reference indicates that the subject is responsive to the anti-cancer therapy. In one embodiment, an increase or no change in the level of biomarker as compared to a reference indicates that the subject is non-responsive to the anti-cancer therapy.

In one embodiment, there is provided a method of monitoring the responsiveness of a subject suffering from lung cancer to an anti-cancer therapy, the method comprising determining the level of a biomarker selected from the group consisting of CAT, CXCR4, SOD3 and SFTPB in a vesicle population isolated from a biological sample from said subject, wherein a change in the level of biomarker as compared to a reference indicates that the subject is responsive to the anti-cancer therapy.

In one embodiment, the method comprises determining the levels of CAT, CXCR4 and SFTPB.

In one embodiment, there is provided a method of monitoring the responsiveness of a subject suffering from lung cancer to an anti-cancer therapy, the method comprising determining the level of a biomarker selected from the group consisting of CAT, CXCR4, SOD3 and SFTPB in a vesicle population isolated from a biological sample from said subject, wherein (a) an increase or no change in the level of biomarker as compared to a reference indicates that the subject is non-responsive to the anti-cancer therapy, and wherein (b) a decrease in the level of biomarker as compared to a reference indicates that the subject is responsive to the anti-cancer therapy.

Provided herein are also compositions for detecting lung cancer in a subject. The composition may comprise an antibody that binds specifically to a protein or peptide biomarker selected from the group consisting of CAT, CXCR4, SOD3 and SFTPB. The antibody may optionally be conjugated to a detectable label.

The compositions as described herein may further comprise a vesicle population isolated from a biological sample from a subject, such as a subject suffering from lung cancer. The vesicle population may optionally be a lysed vesicle population.

Also disclosed herein is the use of a composition as defined herein for detecting lung cancer in a subject.

Provided herein is also a kit for detecting lung cancer in a subject. The kit may comprise an antibody that binds specifically to a protein or peptide biomarker selected from the group consisting of CAT, CXCR4, SOD3 and SFTPB. In one embodiment, the kit comprises antibodies that binds specifically to CAT, CXCR4 and SFTPB. The kit may comprise a suitable buffer for detecting lung cancer in a subject. The kit may comprise components for isolating a vesicle population from a biological sample from a subject. The kit may further comprise a vesicle population isolated from a biological sample from a subject, such as a subject suffering from lung cancer. The vesicle population may optionally be a lysed vesicle population.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1

### Tandem Mass Tag (TMT)-Based Quantitation Workflow for Plasma Exosome Proteome Analyses.

Circulatory exosomes are highly sought after bioentities given their involvement and relevance in virtually every pathophysiological aspect in humans. In the discovery phase, plasma exosomes were isolated using prolong ultracentrifugation (PUC) (1) approach, reported to be effective in simplifying plasma complexity. TMT, a chemical labeling approach that affords both quantitation and multiplexing analyses in a single reagent (2), was employed to establish the differential proteome of pooled plasma exosomes from early-stage NSCLC; late-stage NSCLC and healthy individuals. Briefly, for each sample group, equal concentration of pooled plasma exosome proteins were proteolytically digested with trypsin. Tryptic peptides from each respective groups were labeled with one of the isobaric tags, followed by first dimensional fractionation using weak anion-exchange chromatography. Fractionated labeled peptides were analyzed by LC-MS/MS and relative abundance of specific peptide among sample is determined by comparing the intensities of the TMT reporter fragment ion in the 126-131 m/z region of the peptide product ion spectra. Two biological replicates and three technical replicates were performed to increase the reliability of differences in quantitative changes in protein expression. Differential quantitative proteomics analyses were performed using open source public tools, and differentially expressed (p<0.05) targeted proteins of interested were further scrutinized through extensive literature mining, and based on their novelty and association with cancer progression.

In view of the ease and practicability for sample preparation in both verification and validation phases, exosomes isolation was performed using a commercial exosome isolation kit from Invitrogen. Verification analyses of shortlisted target exosomal proteins by western immunoblotting were performed in a subset of individuals from an orthogonal cohort. Proteins with expressions significantly associated with both early- and late-stage NSCLCs, independent of disease staging were prioritized for validation. Enzyme-linked immunosorbent assay (ELISA) validation of the verified candidates in exosome content and in soluble plasma, were performed alongside with well-established cancer biomarkers in a larger patient cohort. The diagnostic efficiency of the validated exosome markers was evaluated based on the established area under the curve (AUC) obtained from receiver operating characteristic (ROC) curve analyses. Finally, multivariate statistical algorithms were employed to determine the predictive value of the multiprotein signature panel in discriminating NSCLC from non-cancer individuals.

### 2) Characterization of Plasma Exosomes.

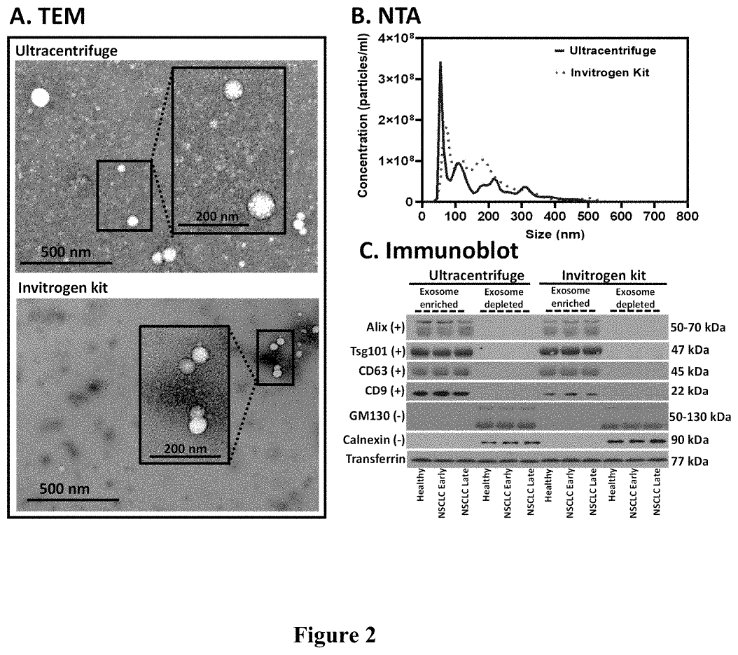

Plasma exosomes-enriched preparations obtained from both ultracentrifugation (UC) and total exosome isolation kit (Invitrogen) were assessed by TEM, NTA and immunoblot analyses in compliance with international guidelines for exosome characterization. TEM analyses (FIG. 2A) on both exosome-enriched preparations showed coexistence of single and aggregated clusters of membrane-bounded spheroidal vesicles, of sizes from 50-150 nm in diameter, consistent to typical characteristics of exosomes. NTA shows the average size distribution for both exosome-enriched preparations (FIG. 2B), with particle sizes ranging from 40 to 500 nm in diameter, which is the expected size range for exosomes (50-150 nm) and small microvesicles (150-1000 nm). The main sizes (mode) of the particles detected from UC and Invitrogen kit preparations were 56.6±1.3 nm and 69.6±2.1 nm, respectively, within the accepted size range of exosomes. The concentration of exosomes enriched from UC and Invitrogen kit were 1.74×10⁹±3.00×10⁸ particles/ml and 2.23×10⁹±8.00×10⁷ particles/ml, accordingly.

The successful recovery of exosomes from both methods were ascertained by immunoblot detection of four common exosome-specific markers, including cytosolic markers Alix and TSG101 and surface markers CD63 and CD9, along with their absence in depleted plasma preparations following exosome extraction (FIG. 2C). Intracellular proteins GM130 and calnexin were selected as negative exosome markers for purity evaluation, absence of the respective markers in both exosome-enriched isolates illustrate the lack of golgi and endoplasmic reticulum (ER) contamination, while both negative markers were detected in all exosome-depleted plasma preparations as expected. However, contaminations from other cellular organelles and vesicles cannot be excluded from both exosome-enriched isolates.

Collectively, these results corroborate that plasma-derived exosomes were highly enriched, with low cell organelle contamination using both UC and Invitrogen methods. UC and Invitrogen exosome isolation methods were employed for exosome enrichment in the discovery phase and verification/validation phases, respectively.

### 3) Assessment of Quantitative MS Data Quality.

TABLE I
Summary of protein, peptide to spectral matches
(PSM) (FDR < 1%) and experimental
variation.

| Identification | TMT_R01 | TMT_R02 | TMT_R03 | SD | % CV | Mean |
|---|---|---|---|---|---|---|
| Protein(s) | 643 | 664 | 652 | 10.54 | 1.61 | 653 |
| PSM (s) | 3968 | 4027 | 4037 | 37.29 | 0.93 | 4011 |

Abbreviation(s): R01, Replicate 01; R02, Replicate 02; R03, Replicate 03; SD, Standard deviation; % CV, Percentage coefficient variation

The run-to-run technical variation was determined in terms of percentage coefficient variation (% CV), the number of proteins and PSMs identified in replicate 01 (R01), replicate 02 (R02) and replicate 03 (R03) were compared as summarized in Table 1. Using a strict FDR<1%, the overall % CV in repeated identification of proteins and PSMs observed across all triplicates were <2%, which translate to minimal run-to-run technical variation and good system repeatability.

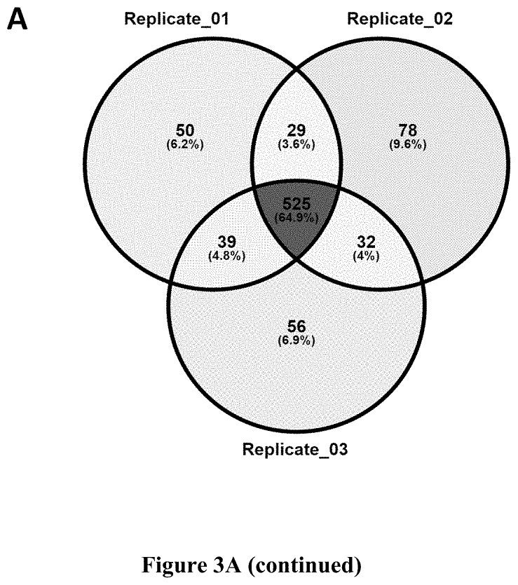

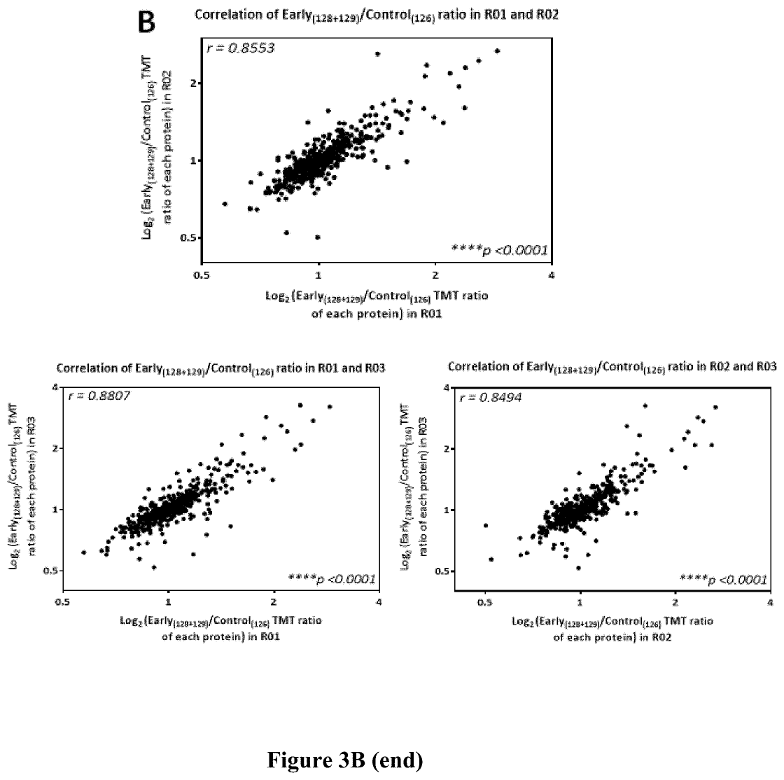

Depicted in FIG. 3A, approximately 77% (625) of the total number of proteins were quantified in at least two of the triplicates, suggesting good protein complementation between the triplicates LC-MS/MS runs, and these proteins were used for further analyses. In FIG. 3B, significant (p<0.05) correlation of measured pairwise Early:Control relative ratios for each protein between triplicates confirmed the reliability and confidence of the quantitative dataset.

### 4) Biomarker Candidate Selection Criteria and Verification Analyses.

TABLE II
Identified list of currently available biomarkers for cancer.

| Accession | Description | GS | Cov | Peptides (95%) | Early:Ctrl | Late:Ctrl |
|---|---|---|---|---|---|---|
| M0R2Y5 | Mucin-16 | CA-125 | 8.21% | 3 | 1.06 | 1.04 |
| P21860 | Receptor tyrosine-protein kinase erbB-3 | ERBB3 | 0.75% | 2 | 0.92 | 1.01 |
| Q3KRG8 | Carcinoembryonic antigen-related cell adhesion molecule 1 | CEACAMI | 5.07% | 2 | 1.09 | 1.06 |
| M0QX98 | Carcinoembryonic antigen-related cell adhesion molecule 5 | CEACAM5 | 26.24% | 2 | 1.20 | 1.22 |

Abbreviation(s): GS, Gene Symbol; Cov, Protein Coverage

Herein, known lung cancer biomarkers identified in the proteomics dataset are listed in Table 3. Although the following markers are clinically available, they are currently used in a limited capacity as accessory blood biomarkers for lung cancer. Based on the fold-change reference of these proteins, 1.2-fold change cut-off was considered as altered expression in this study. Next, to further refine the list of candidate markers, differentially expressed candidates were selected based on the following criteria: (a) protein must be identified based on ≥2 peptides with 95% confidence and quantified in at least two of the triplicates; (b) Proteins have to display at least 1.2-fold change; (c) and only proteins that were significantly (p<0.05) differentially expressed will be considered. Proteins that did not meet these stringent criteria were disregarded. As a result, from the core list of 625 proteins, a total of 56 exosome proteins were found to display concurrent differential regulation in both NSCLC phenotype relative to controls. These proteins were further scrutinized through extensive literature mining, and based on their novelty and association with cancer progression, ten markers (Table III) were shortlisted for verification by immunoblotting in a subset of individuals (early-stage NSCLC, n=14; late-stage NSCLC, n=14 and healthy individuals, n=14) not used in the discovery experiments.

TABLE III
Significantly (p <0.05) differentially regulated NSCLC-specific candidate proteins.

| | | | | | | Peptides | Early:Ctrl | Late:Ctrl |
|---|---|---|---|---|---|---|---|---|
| No | Score | Accession | Gene Name | Protein Description | Function | (95%) | Ratio | Ratio |
| 1 | 42.36 | B4DF70 | PRDX2 | Peroxiredoxin-2 | Antioxidant | 4 | 1.58 | 1.39 |
| 2 | 22.09 | U3KPZ0 | TPI1 | Triosephosphate isomerase | Isomerase activity | 2 | 1.51 | 1.47 |
| 3 | 31.09 | P05109 | S100A8 | Protein S100-A8 | Calcium binding | 5 | 2.04 | 1.98 |
| 4 | 61.63 | Q08830 | FGL1 | Fibrinogen-like protein 1 | Hemostasis | 6 | 1.39 | 1.90 |
| 5 | 41.11 | D3JV41 | CXCR4 | C-X-C motif chemokine | Heparin-binding | 4 | 2.07 | 1.33 |
| | | | | receptor 4 | | | | |
| 6 | 26.66 | Q8TAK2 | CAT | Catalase | Antioxidant | 7 | 1.38 | 1.36 |
| 7 | 8.94 | B2R9V7 | SOD3 | Superoxide dismutase [Cu-Zn] | Antioxidant | 2 | 1.21 | 1.24 |
| 8 | 159.18 | P00915 | CA1 | Carbonic anhydrase 1 | Zinc metalloenzymes | 11 | 1.61 | 1.47 |
| 9 | 9.4 | PO7737 | PFN1 | Profilin-1 | Actin-binding protein | 2 | 1.32 | 1.39 |
| 10 | 3.52 | PO7988 | SFTPB | Pulmonary surfactant-associated | Lung function and | 2 | 1.43 | 1.82 |
| | | | | protein B (or Surfactant protein | homeostasis | | | |
| | | | | B) | | | | |

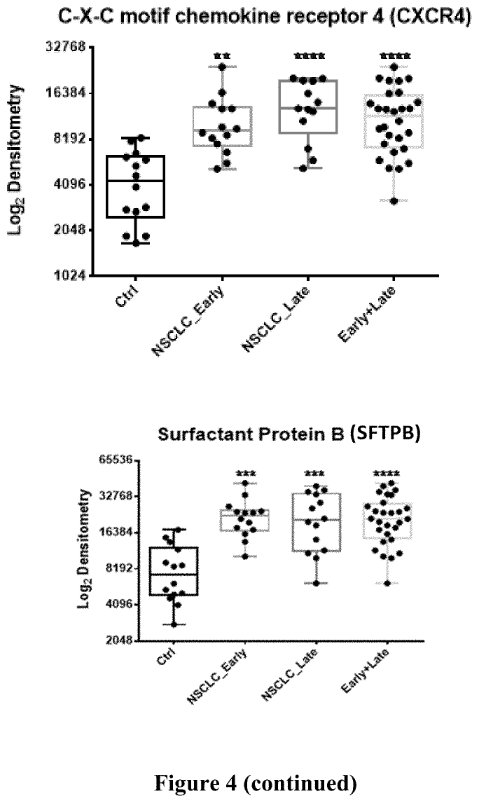

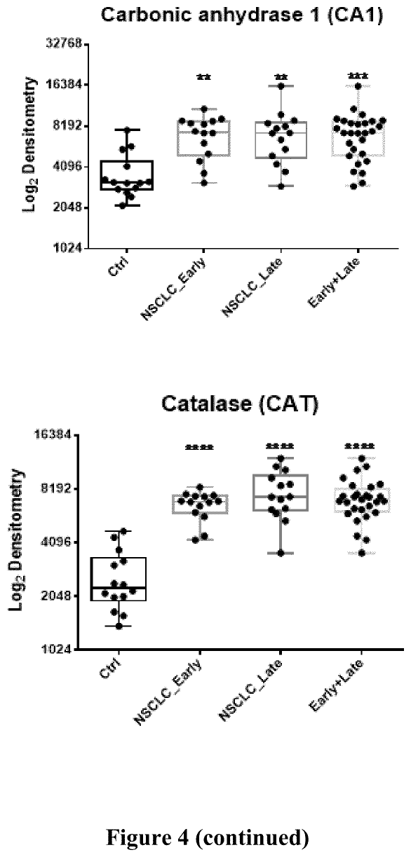

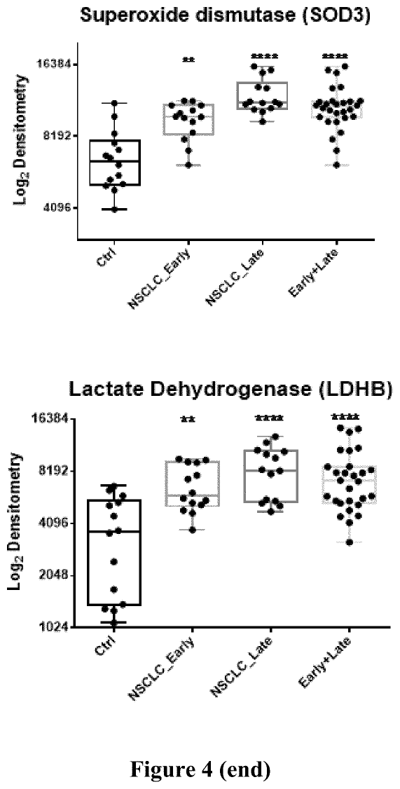

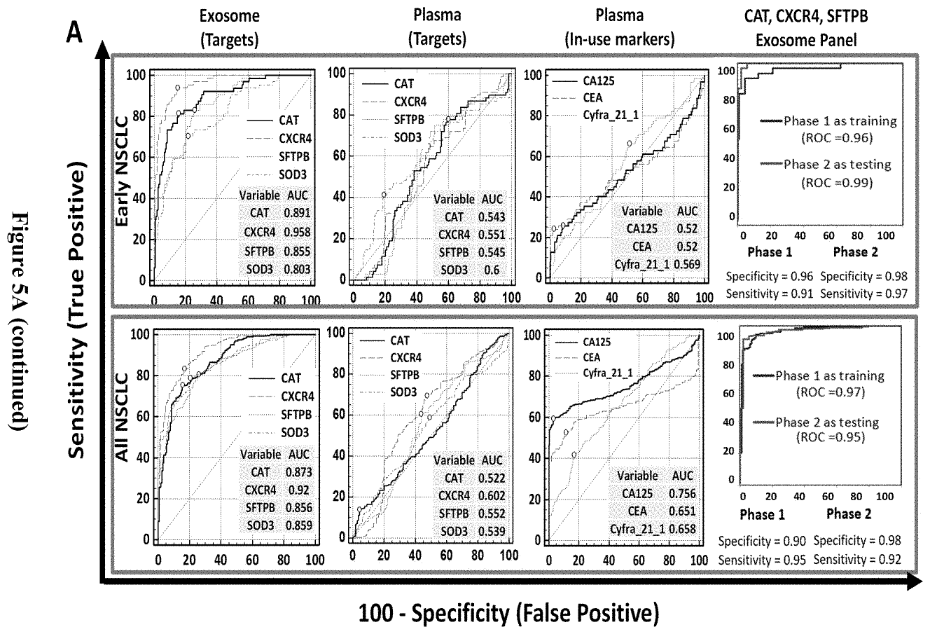

Verification analyses returned with six exosome proteins that were in statistical agreement with discovery proteomics dataset, and with expressions highly associated with both early- and late-stage NSCLC (FIG. 4), and these proteins will move forth into the clinical validation phase. It is emphasized that surfactant protein B (SFTPB), is a lung specific proteins that is only expressed in lung tissues. The strategy in the selection of NSCLC biomarkers lies in that the candidate protein should display concurrent significant increased protein expression (p<0.05) in both early- and late stage NSCLC relative to controls respectively. This would ensure that the candidates can be reliably use to detect early stage NSCLC, and it is shown that increased expression is independent of disease advancement.

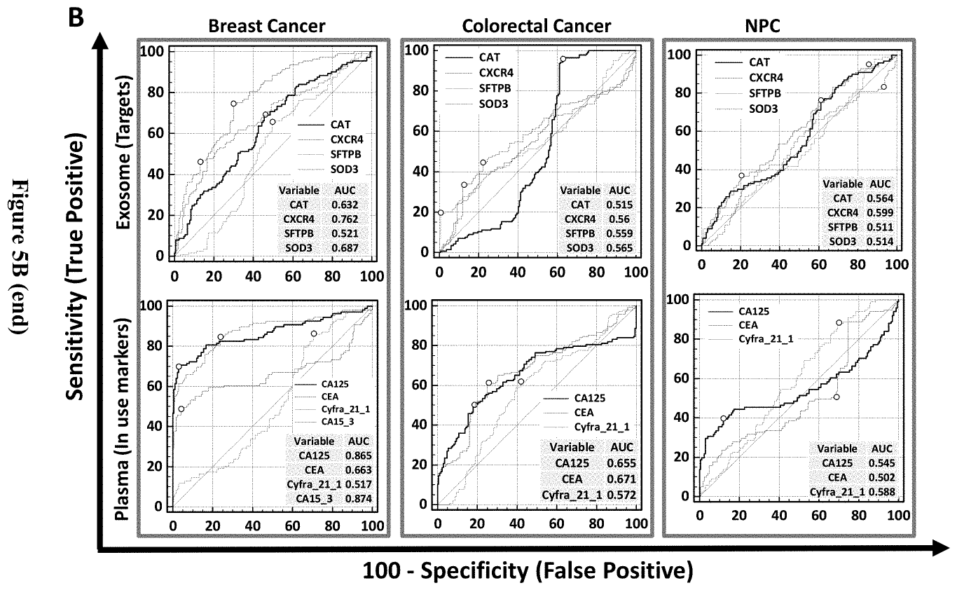

Validation of these six markers were performed using enzyme-linked immunosorbent assay (ELISA) on total of 306 individuals (early-stage NSCLC patients (n=53); late-stage NSCLC patients (n=139) and healthy individuals (n=114). Among the six candidates, four markers (CAT, CXCR4, SOD3, SFTPB) displayed similar significant (p<0.05) differential expression between healthy subjects and NSCLC phenotypes, as reported in both discovery and verification phase, and were further evaluated using receiver operating characteristic (ROC) curves. ROC curves based on the ELISA results were plotted to compare the diagnostics efficiency of the four candidate markers, alongside with 2 well-studied cancer markers, in both exosomal content and in soluble plasma. The discriminatory capacity of each individual candidate, and the 4-marker combined panel, between healthy controls and early-stage NSCLC (FIG. 5A)/all NSCLC cases were evaluated using AUC under the ROC curves. The four-marker combined panel displayed the highest ROC AUC value of 0.93, in discriminating NSCLCs from non-cancer controls. Compared to well-studied cancer biomarkers (CEA, Cyfra21), the four-marker panel and all standalone markers possessed greater efficacy for the diagnosis of NSCLCs. This is the first lung cancer study that compares the target cargo in exosomes with their respective soluble levels in patients' plasma. These four biomarkers have been associated with cancer progression, with CAT, SOD3 and SFTPB conferring anti-tumorigenic functions, and CXCR4 harboring pro-tumorigenic functions.

### 5) Clinical Validation

In clinical validation, patients were approximately split into half and assigned into a training group (n=279) and validation test set (n=305) for Phase I and Phase II validation (Table IV), respectively. In Phase I validation, data obtained from the training set are used to train the multivariate model to give a combined receiver operator curve (ROC) analyses or predictive value of the final signature panel in discriminating NSCLC from non-cancer individuals. In Phase II validation, data obtained from the test set are used to validate the trained model.

TABLE IV
Phase I and II datasets used in the derivation
of exosome multivariate predictive model.

| Exosome Targets | Phase I Training Set (n) | Phase II Test Set (n) |
|---|---|---|
| Healthy | 114 | 53 |
| Early NSCLC | 32 | 32 |
| All NSCLC | 133 | 220 |

Abbreviation(s): NSCLC, Non-small cell lung cancer; n, sample size.

The initial six candidates selected in verification phase were evaluated on the training set samples (early-stage NSCLC patients (n=32); all NSCLC patients (n=133) and healthy individuals (n=114). using enzyme-linked immunosorbent assay (ELISA). Among the six candidates, four markers (CAT, CXCR4, SOD3, SFTPB) displayed similar significant (p<0.05) differential expression between healthy subjects and NSCLC phenotypes, as reported in both discovery and verification phase. The Phase I validated data obtained from these four exosome targets was subjected to in-house machine learning multivariate model training to finally derive a three-marker signature panel (CAT, CXCR4, SFTPB) for early NSCLC (AUC=0.96; specificity=0.96; sensitivity=0.91). With no adjustment to the model, Phase II validation data obtained from the test set samples will be used to cross evaluate the prediction of the model

In FIG. 5A, validated resulted obtained from Phase I and Phase II were used to plot the ROC to evaluate and compare the diagnostics efficiency of the four candidates, alongside with three well-studied cancer markers (CA125, CEA, Cyfra-21), in both exosomal content and in soluble plasma of matching patients. The discriminatory capacity of each individual candidate, and the three-marker signature panel, between healthy controls, early-stage NSCLC and all NSCLC cases were evaluated using AUC under the ROC curves. On each of the four exosome markers, a total of 167 healthy individuals and 353 NSCLC patients were validated, where 64 are early cases, and it was shown that all markers showed better predictive value in exosome contents and not in plasma. With the inclusion of Phase II validation data, the three-marker signature panel displayed the highest ROC AUC value of 0.99 (specificity=0.98; sensitivity=0.97) in discriminating early NSCLCs from non-cancer controls. In comparison to the three well-studied cancer markers (CA125, CEA and Cyfra-21), all standalone markers and the three-marker signature panel possessed greater efficacy for the diagnosis of NSCLCs.

In addition, ELISA validation on each four exosome marker and well-studied cancer markers was performed on breast cancer (n=113), colorectal cancer(n=144) and nasopharyngeal cancer (NPC) (n=101) groups, covering the top 3 common global cancer types (FIG. 5B). In comparison to well-studied cancer markers (CA125, CEA, Cyfra-21), the standalone four markers showed almost no discriminatory capacity, with AUC ranging from 0.5 to 0.6 for breast cancer, colorectal cancer and NPC, suggesting that the four markers are indeed specific to NSCLC diagnosis. It is also shown that the different cancer phenotypes are indeed true to as claimed, with all three cancer phenotype scoring AUC of approximately 0.5 for Cyfra21-1 (lung marker), breast cancer scoring AUC of 0.877 for CA 15-3 (breast marker), colorectal cancer scoring AUC of 0.671 (colorectal marker), and NPC showing no discriminatory capacity for all well-studied cancer-markers, as expected.

The three exosome targets in the signature panel have been associated with cancer progression, with CAT and SFTPB conferring anti-tumorigenic functions, and CXCR4 harboring pro-tumorigenic functions. In combination, the three-marker exosome signature have immense clinical utility in the diagnosis of heterogeneous NSCLC.

1.-17. (canceled)

18. A method of treating a lung cancer in a subject, the method comprising (a) determining the levels of the biomarkers, catalase (CAT) and C-X-C motif chemokine receptor 4 (CXCR4), from a vesicle population isolated from a biological sample from said subject, wherein a change in the levels of the biomarkers as compared to a reference indicates the likelihood of the presence of lung cancer in the subject; and (b) administering an anti-cancer therapy to the subject when the subject is determined to have a likelihood of the presence of lung cancer.

19. The method of claim 18, wherein an increase in the levels of the biomarkers as compared to a reference indicates the likelihood of the presence of lung cancer in the subject.

20. The method of claim 18, wherein the biomarkers are nucleic acids, proteins or peptides.

21. The method of claim 18, wherein the vesicle is an extracellular vesicle.

22. The method of claim 18, wherein the levels of the biomarkers are determined using antibody-based techniques or PCR-based techniques.

23. The method of claim 18, wherein the method comprises isolating the vesicle population from said biological sample.

24. The method of claim 18, wherein the method comprises detecting the vesicle population with an antibody.

25. The method of claim 24, wherein the antibody is selected from the group consisting of an anti-CD9 antibody, an anti-CD63 antibody and an anti-CD81 antibody.

26. The method of claim 18, wherein the biological sample is a liquid biopsy.

27. The method of claim 18, wherein the cancer is non-small cell lung cancer (NSCLC) or small cell lung cancer (SCLC).

28. The method of claim 27, wherein the NSCLC or SCLC is early NSCLC or early SCLC.

29. The method of claim 18, wherein the method further comprises performing a chest computed tomography (CT) on the subject.

30. The method of claim 18, wherein the method comprises detecting the levels of CAT, CXCR4 and surfactant protein B (SFTPB).

31. A method of detecting and treating lung cancer in a subject, the method comprising: (a) determining the levels of the biomarkers, CAT and CXCR4, in a vesicle population isolated from a biological sample from said subject, wherein a change in the levels of the biomarkers as compared to a reference indicates the presence of lung cancer in the subject, and (b) administering an anti-cancer therapy to the subject when the subject is found to have lung cancer.

32. A method of treating a lung cancer in a subject, the method comprising (a) selecting a subject based on the levels of the biomarkers, CAT and CXCR4, in a vesicle population isolated from a biological sample from said subject, wherein a change in the levels of the biomarkers as compared to a reference indicates the presence of lung cancer in the subject; and b) administering an anti-cancer therapy to the subject when the subject is found to have lung cancer.
